(19) United States
(12) Patent Application Publication
(10) Pub. No.: US 2010/0145170 A1
(43) Pub. Date: Jun. 10, 2010

(54) Small Animal Pulse Oximeter User Interface

(75) Inventors: Eric J. Ayers, Aliquippa, PA (US); Bernard F. Hete, Kittanning, PA (US); Patrick W. Truitt, Murrysville, PA (US)

Correspondence Address: BLYNN L. SHIDELER;THE BLK LAW GROUP, 3500 BROKKTREE ROAD, SUITE 200, WEXFORD, PA, 15090, US

(73) Assignee: STARR LIFE SCIENCES CORP., Oakmont, PA (US)

(21) Appl. No.: 12/473,244

(22) Filed: May 27, 2009

Related U.S. Application Data
(63) Continuation-in-part of application No. 11/972,431, filed on Jan. 10, 2008.
Application No. 11/972,431 is a continuation-in-part of application No. 11/858,877, filed on Sep. 20, 2007.
Application No. 11/972,431 is a continuation-in-part of application No. 11/951,194, filed on Dec. 5, 2007.
(60) Provisional application No. 61/056,092, filed on May 27, 2008.
(60) Provisional application No. 60/884,392, filed on Jan. 10, 2007.
(60) Provisional application No. 60/826,530, filed on Sep. 21, 2006.
(60) Provisional application No. 60/868,681, filed on Dec. 5, 2006.
(60) Provisional application No. 60/884,392, filed on Jan. 10, 2007.

(51) Int. Cl. A61B 5/1455

(52) U.S. Cl. 600/324

(57) ABSTRACT

A user interface for a pulse oximetry device that calculates physiologic parameters of a subject including at least a subject's heart rate and SₚO₂, is disclosed wherein the interface comprises a graphical display of at least one raw data signal of the pulse oximetry device that maintains heart and breath rate components and a display of the calculated heart rate and SₚO₂ of the subject. The interface may further include a user selectable data averaging function in which the interface is configured to selectively obtain and display averages of at least some of the calculated physiologic parameters over a defined period. The interface may further include a user selectable noise suppression function for the displayed data.

### RELATED APPLICATIONS

This application claims the benefit of U.S. Provisional patent application Ser. No. 61/056,092, filed May 27, 2008 entitled “Small Animal Pulse Oximeter User Interface.”

This application is a continuation in part of U.S. patent application Ser. No. 11/972,431 filed Jan. 10, 2008 entitled “Small Animal Pulse Oximeter User Interface”. U.S. patent application Ser. No. 11/972,431 claims the benefit of U.S. Provisional patent application Ser. No. 60/884,392 filed Jan. 10, 2007 entitled “Small Animal Pulse Oximeter User Interface.”

U.S. patent application Ser. No. 11/972,431 is a continuation in part of U.S. patent application Ser. No. 11/858,877 filed Sep. 20, 2007 entitled “Medical Display Devices for Deriving Cardiac and Breathing Parameters Derived from Extra-thoracic Blood Flow Measurements.” Application Ser. No. 11/858,877 claims the benefit of provisional patent application Ser. No. 60/826,530 entitled “Medical Devices and Techniques for Deriving Cardiac and Breathing Parameters from Extra-thoracic Blood Flow Measurements and for Controlling Anesthesia Levels and Ventilation Levels in Subjects” filed Sep. 21, 2006.

U.S. patent application Ser. No. 11/972,431 is a continuation in part of U.S. patent application Ser. No. 11/951,194 filed Dec. 5, 2007 entitled “Research Data Classification and Quality Control for Data from Non-Invasive Physiologic Sensors.” Application Ser. No. 11/951,194 claims the benefit of U.S. Provisional patent application Ser. No. 60/868,681 filed Dec. 5, 2006 entitled “Research Data Quality Control Software.” Application Ser. No. 11/951,194 claims the benefit of U.S. Provisional patent application Ser. No. 60/884,392 filed Jan. 10, 2007 entitled “Small Animal Pulse Oximeter User Interface.”

### BACKGROUND OF THE INVENTION

1. Field of the Invention

The present invention relates to user interface for physiologic parameter sensors and more particularly to small animal pulse oximeter user interfaces.

2. Background Information

The present invention is related to the user interface provided in physiologic sensor devices, particularly those for use with non-invasive physiologic sensors, such as pulse oximeters, and in particular those used on small subjects in a research environment.

As background, one type of non-invasive physiologic sensor is a pulse monitor, also called a photoplethysmograph, which typically incorporates an incandescent lamp or light emitting diode (LED) to trans-illuminate an area of the subject, e.g. an appendage, which contains a sufficient amount of blood. The light from the light source disperses throughout the appendage {which is broken down into non-arterial blood components, non-pulsatile arterial blood, and pulsatile blood}. A light detector, such as a photodiode, is placed on the opposite side of the appendage to record the received light. Due to the absorption of light by the appendage's tissues and blood, the intensity of light received by the photodiode is less than the intensity of light transmitted by the light source (e.g., LED). Of the light that is received, only a small portion (that effected by pulsatile arterial blood), usually only about two percent of the light received, behaves in a pulsatile fashion. The beating heart of the subject, and the breathing of the subject as discussed below, create this pulsatile behavior. The “pulsatile portion light” is the signal of interest, and effectively forms the photoplethysmograph. The absorption described above can be conceptualized as AC and DC components. The arterial vessels change in size with the beating of the heart and the breathing of the patient. The change in arterial vessel size causes the path length of light to change from dₘᵢₙ to dₘₐₓ. This change in path length produces the AC signal on the photo-detector, which spans the intensity range, I_{L} to I_{H}. The AC Signal is, therefore, also known as the photoplethysmograph.

The absorption of certain wavelengths of light is also related to oxygen saturation levels of the hemoglobin in the blood transfusing the illuminated tissue. In a similar manner to the pulse monitoring, the variation in the light absorption caused by the change in oxygen saturation of the blood allows for the sensors to provide a direct measurement of arterial oxygen saturation, and when used in this context, the devices are known as oximeters. The use of such sensors for both pulse monitoring and oxygenation monitoring is known, and in such typical uses, the devices are often referred to as pulse oximeters. These devices are well known for use in humans and large mammals and are described in U.S. Pat. Nos. 4,621,643; 4,700,708 and 4,830,014, which are incorporated herein by reference.

Current commercial pulse oximeters do not have the capability to measure breath rate or other breathing-related parameters other than blood oxygenation. An indirect (i.e. not positioned within the airway or air-stream of the subject), non-invasive method for measuring breath rate is with impedance belts. Further, prior to the implementation of the MouseOx™ brand pulse oximeter, introduced in mid-December 2005, there were no commercial pulse oximeters that were effective for small mammals such as mice and rats.

These existing physiologic sensor devices, particularly those for use with small subjects in a research environment, need a user interface to display results to the user and to further allow the user to effectively utilize the sensor devices. In general, many existing sensor device merely have a display to display current readings to the user, and the only functional system controls are the on/off controls. This limited user interface restricts the uses for the sensor device, particularly in a research environment.

It is an object of the present invention to minimize the drawbacks of the existing technology and to provide a simple easy to use small animal physiologic sensor user interface.

### SUMMARY OF THE INVENTION

The present invention is directed toward the user interface for a physiologic parameter sensor that calculates physiologic parameters of a subject, such as a pulse oximeter. The details of the pulse oximeter, per se, and other physiologic parameter sensors (blood pressure monitors, eeg, ekg etc) are known in the art and not discussed herein in detail. The present invention is directed to the interface that allows the user, particularly a researcher, to more efficiently and effectively implement these sensor tools.

One non-limiting embodiment of the present invention provides a user interface for a pulse oximetry device that calculates physiologic parameters of a subject including at least a subject's heart rate and SₚO₂, wherein the interface comprises a graphical display of at least one raw data signal of the pulse oximetry device that maintains heart and breath rate components and a display of the calculated heart rate and SₚO₂ of the subject. The phrase “raw data signal” with regards to pulse oximetry devices will mean, within this application, a signal that maintains the heart and breath components of the signal together. The “raw” signal will typically undergo some signal processing (also called pre-processing such as analog filters and gains), but such processing is minimal and this signal is therefore considered raw within this application. This raw signal exhibits a much faster real time response than do the processed breath and heart rate signals.

The pulse oximeter user interface of the present invention may further include a graphical display of a plurality of the calculated physiologic parameters over time, and a numerical display of a plurality of the calculated physiologic parameters at selected times, such as at the most recent calculation and/or at a user designated time.

The pulse oximeter user interface of the present invention further includes a recording of the calculated physiologic parameters and an event file marker function which is configured to be user selected to physically identify selected time locations of the record. The file marker function may physically identify the selected times on an associated graphical display of the calculated physiologic parameters and may further mark a location of a recorded session.

One non-limiting embodiment of the present invention provides a user interface for a physiologic parameter sensor that calculates physiologic parameters of a subject, wherein the interface comprises a user selectable data averaging function in which the interface is configured to selectively obtain and display averages of at least some of the calculated physiologic parameters over a defined period.

The physiologic parameter sensor user interface of the present invention may provide that the data averaging further includes a user selection of the defined average period. The data averaging may be configured to ignore calculated physiologic values that are deemed unacceptable in the calculation of the averages. The data averaging may be configured to display intermediate average values during calculation and to display final average values to the user in a distinct manner from the display of the intermediate average values. The interface may be configured to selectively begin the defined period at any time designated by the use. The interface may be configured to operate on recorded or real time data.

These and other advantages of the present invention will be clarified in the description of the preferred embodiments taken together with the attached figures in which like reference numerals represent like elements throughout.

### BRIEF DESCRIPTION OF THE FIGURES

FIG. 1 is a representative illustration of a summary data screen for a pulse oximeter user interface according to one embodiment of the present invention;

FIG. 2 is a representative illustration of a more detailed summary data screen for the pulse oximeter user interface of FIG. 1;

FIG. 3 is another representative illustration of the summary data screen of FIG. 2;

FIG. 4 is a representative illustration of a main data collection screen for the pulse oximeter user interface of FIG. 1;

FIG. 5 is another representative illustration of the main data collection screen of FIG. 4;

FIG. 6 is another representative illustration of user selectable data averaging diagnostic screen for the pulse oximeter user interface of FIG. 1;

FIGS. 7a-c are representative illustrations of another version of a main data collection screen with user defined noise suppression function; and

FIG. 8 is another representative illustration of user selectable data averaging diagnostic screen for the pulse oximeter user interface of FIG. 1.

### DESCRIPTION OF THE PREFERRED EMBODIMENTS

The present invention is directed to the user interface of a physiologic sensor device, such as a pulse oximeter device, particularly a physiologic sensor device for small mammals, such as found in many research applications. In such devices the output, generally including a display of the sensed parameter as determined by the sensor device, is displayed to the user in some format on an associated display device. The details of the physiologic sensor device are known in the art and are not included herein. The present invention has been implemented as a user interface on the MouseOx™ brand pulse oximeter for small animals, such as rats and mice. The invention can be implemented on other brands of pulse oximeters and other physiologic sensors. The advantages of the present invention are most notable in a research environment, but the invention is not limited thereto. Similarly, much research is done on animals, and the largest majority of animal research is performed with mice and rats. The present invention is clearly well suited for such animal research applications, but it is not limited to use with animal related sensors.

Pulse Pleth Window

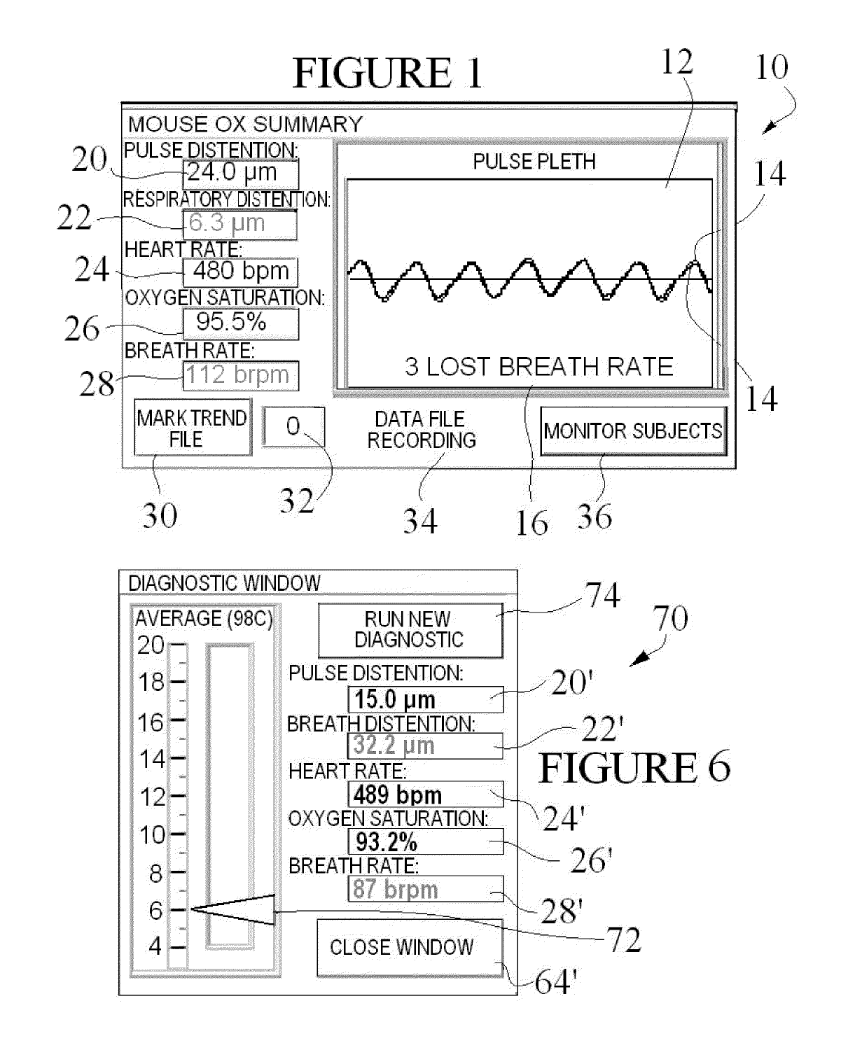

The first aspect of the present invention is shown on a summary screen 10 of the interface of the present invention shown in FIG. 1. The summary screen includes a window 12 referenced as the Pulse Pleth window 12. The window 12 appears on the pulse oximeter summary screen 10, the detailed summary screen 40 (described below) and the main data collection screen 50 (described below) in the Mouse Ox™ device sold by Starr Life Sciences, and provides a near real-time graphical display of the transmitted red and infrared pulse oximeter light intensities 14 as received by the receiver, to the user. In the manifestation as shown in the figure, the display 12 appears as dual oscilloscope traces 14. A red trace 14 represents the red transmitted light intensity, while a yellow trace 14 represents the infrared transmitted light intensity. The transmitted light data that form these traces 14 are received in packets from the A/D card buffer and are transmitted across the USB cable to the computer. Once in the computer, they are processed in various ways and sent to the Pulse Pleth window 12 for graphical display. The Pulse Pleth window 12 as it appears in the MouseOx™ Summary screen 10 is what is shown in FIG. 1.

One important utility of this graphical representation in window 12 of what is effectively raw data is that it allows the user to see the waveforms 14 so that their quality can be judged. Since the quality of the waveforms 14 determines the ability of the pulse oximeter to make continuous accurate measurements of its parameters, displaying the “raw signal” traces 14 to the user can allow him to be able to move/adjust the sensor location in order to improve signal quality. The raw data traces 14 are sufficient feedback for the user to perceive weaker and stronger signals based upon sensor location (within what ever adjustment is provided in a particular sensor mount).

Note that the particular color of the traces 14 is inconsequential, and that the data does not have to be delayed or processed in order to provide beneficial information to the user. Additionally, the processing could be conducted in the same device that has the A/D board and/or the display screen.

Summary Screen

The remaining elements of the summary screen 10 should be discussed for a fuller understanding of the interface of the present invention. The summary screen 10 includes a numerical display of the physiologic parameters measured by the MouseOx™ pulse oximeter. These include a numerical display of the latest pulse distension measurement 20 with associated heading; a numerical display of the latest breath distension measurement 22 with associated heading; a numerical display of the latest heart rate measurement 24 with associated heading; a numerical display of the latest SₚO₂ (oxygen saturation) measurement 26 with associated heading; and a numerical display of the latest breath rate measurement 28 with associated heading.

The summary screen 10 further includes a control button 30 that will mark the data file as will be described in further detail below as it is an important aspect of the interface of the present invention. The summary screen 10 includes a file marker number 32 to indicate to the user which file marker has been set.

The summary screen 10 further includes a status indicator 34 to identify if the system is recording, or playing back a recorded session or idle. Other status indicators can be added as desired. The summary screen 10 can include a variety of other control buttons 36 to perform other designated tasks such as pulling up windows, closing windows, and other interface that is necessary to better implement the oximeter.

Parameter Color Change

An improvement in data error indication involves letting the user know about problems with the data while the data is being collected. Although the quality of data can be assessed in a general sense using the Pulse Pleth window 12 described above, data signals from the Pulse Pleth window 12 that are judged to be of sufficient quality, may still result in the inability for the software algorithms to successfully calculate one or more parameters at a given instant of time. An additional aid to the user has been provided by changing the color of a given parameter in the data text boxes 20-28 each time calculation of the associated parameter in the given text box 20-28 does not pass the acceptance criterion for that parameter. An error flag may be thrown in a log file such cases that allow the user to flag data that is questionable at a later review. Additionally here an indication of a problem 16 is given on the window 12 and possibly on the main user screen while data are being collected. This feedback may be done in two ways. The first is that the background of the Pulse Pleth screen or window 12 changes color from black to green (and note that the color choices are arbitrary) while an error flag is active. Secondly, the numerical values displayed in the data text boxes 20-28 change color, including a color that matches the background of the text box such that the number is not seen, when a given parameter does not pass the acceptance criterion for that parameter. This display utility could be further improved by changing the background color on a given data display plot associated with a given error flag at a given time.

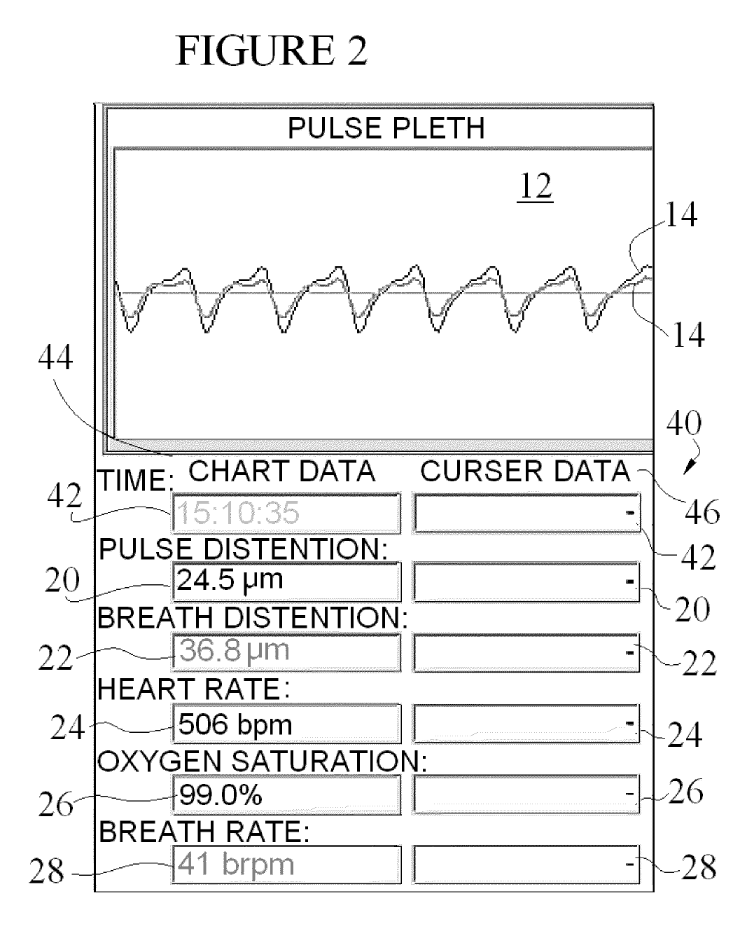

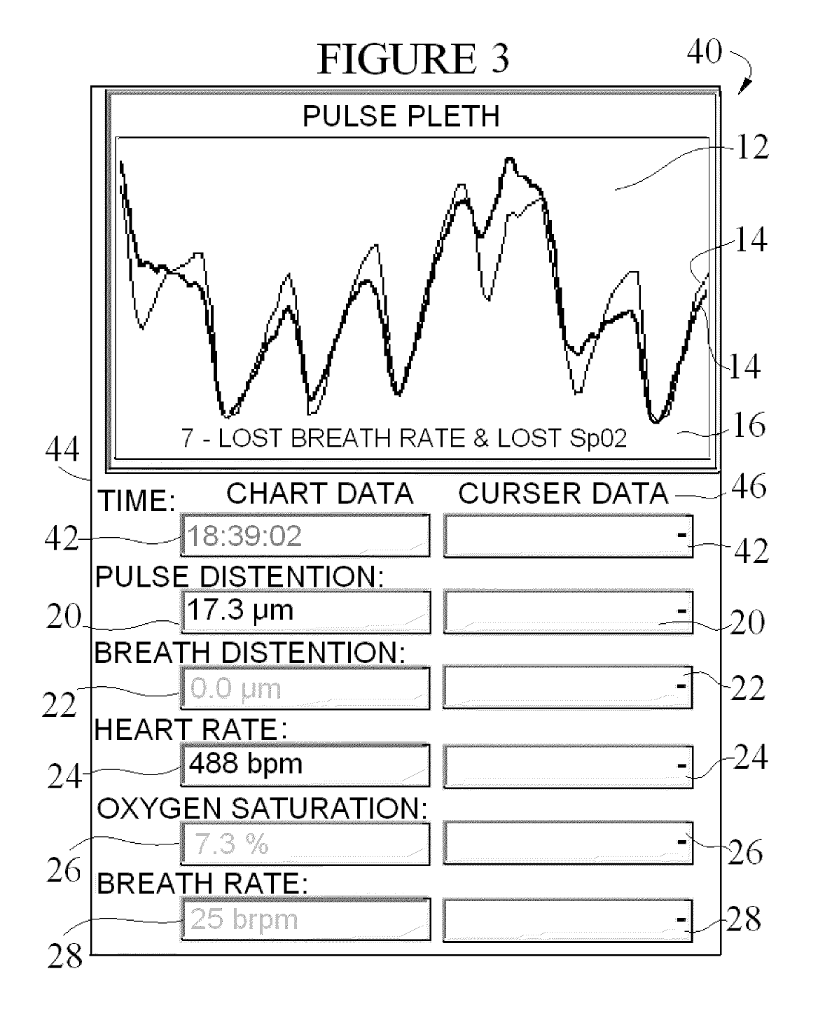

Shown in FIG. 2 a pictorial representation of the detailed summary display 40 of the user interface in normal operation, and FIG. 3 is a representation of the display 40 of the user interface in operation while an error flag 16 is present. Obviously, these are not the same data sets, but serve to illustrate the two different cases. Note that not only the color change in the Pulse Pleth window 12, but also the “graying out” of the Chart Data 22, 26 and 28 (also a color change) for the affected parameters only. Other parameters 20 and 24 are still considered to be valid.

The detailed summary display or window 40 includes the parameter displays 20-28 for the most current data sets under the chart data heading 44. Further the summary window 4 includes a display of the parameters 20-28 at a user selected location, such as at a file marker, under the heading curser data 46. In light of the two sets of data values 20-28 that may be displayed in window 40, a time indicator 42 is included above each column to convey the associated event time that each column is reflecting.

Quick Diagnostic Measurement: Graphical Display of Parameters Over Time

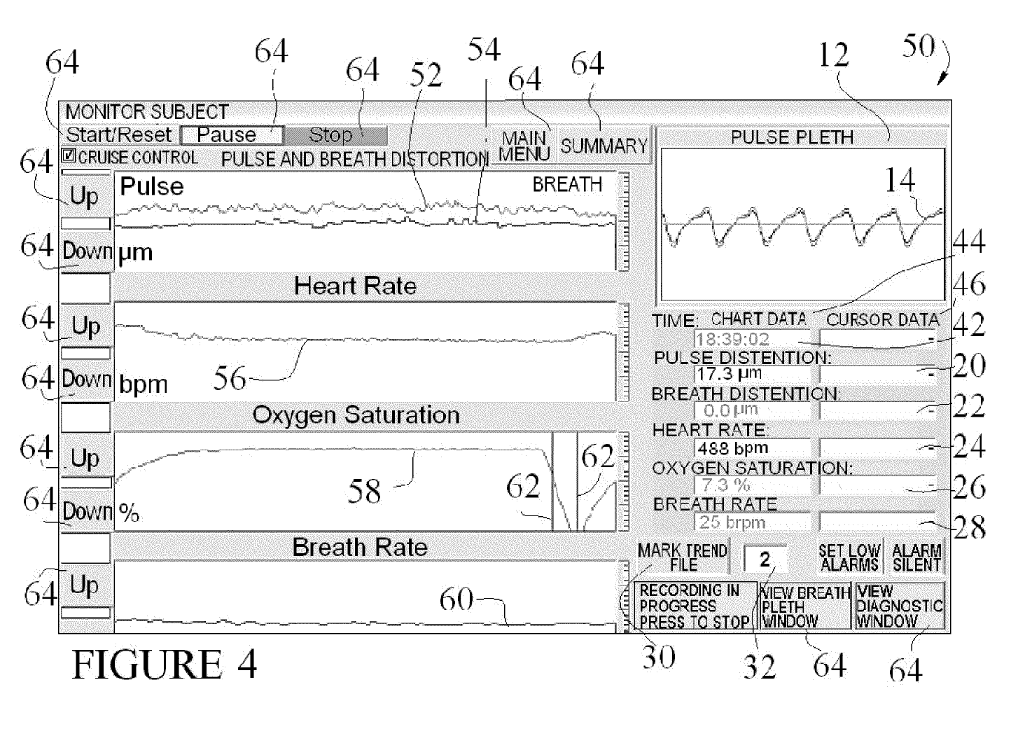

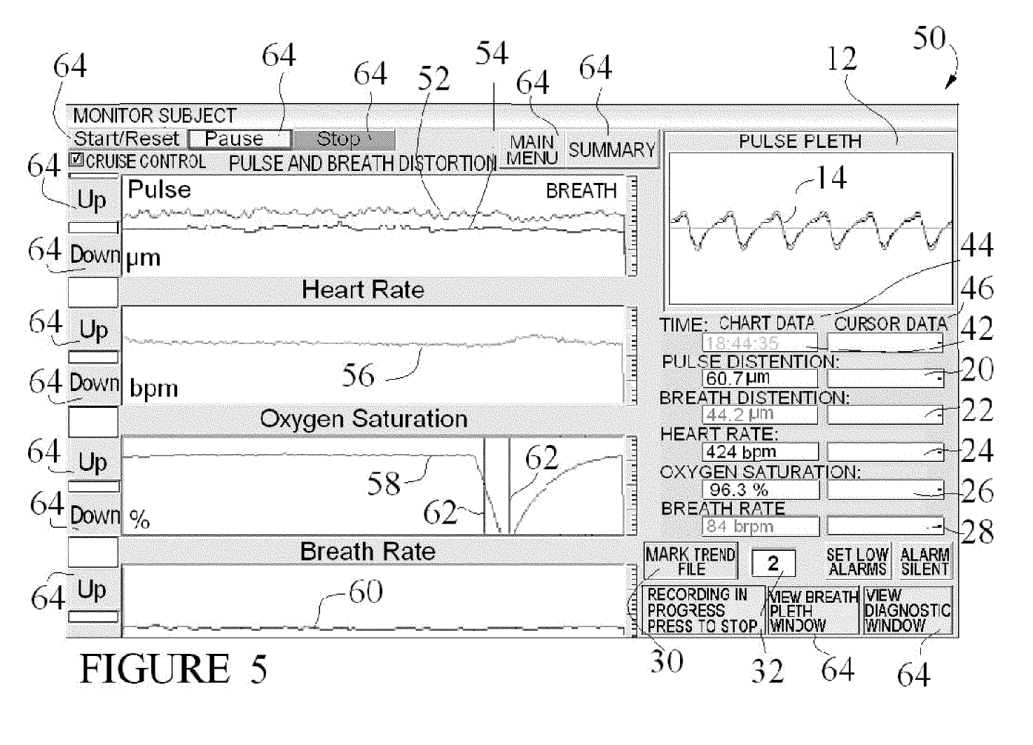

The following concepts deal with improving the ability of a device user to monitor the status of the animal, as well as the progress of a given experiment. The first item is the continuous graphical display of each of the parameters on the main data collection screen 50, as well as the off-line data review screen (not shown, but is substantially the same as 50 with playback controls 36). These graphically displayed parameters include heart rate 56, breath rate 60, SₚO₂ 58, pulse distention 52 and breath distention 54. Graphs could also be added to include any parameters that may be developed in the future. The graphs 52-60 consist of continuous streaming plots of each parameter. The graphs are displayed on a data point basis, which can be considered a time based display, however technically the displays would be display a range of data points with the data points evenly distributed. As the range of data points corresponds to a range of time it is essentially a time based display. Because of the time-based display of these graphs 52-60, they also allow the user to watch the response to a given input in an experiment. These graphical displays can be seen on the left-hand side of the display 50 of FIGS. 4 and 5. In the particular embodiment the sensor is a pulse oximeter such as sold under the brand Mouse Ox by Starr Life Sciences.

The main data collection screen or display 50 of the interface of the present invention further includes the window 12, and numerical displays 20-28 for the current data (chart data 44) and at a user selected location (curser data 46), and file marking control 30, and numerical file marker indicator 32, and a series of additional controls 64 for interfacing with the display 50. The controls 64 include buttons to stop/start and pause the recording session and to bring up other displays, to close a display, and increase/decrease the visible gain on a selected graph. Other controls 64 can be added as interface further functions are desired.

File Marker

Associated with this benefit is the ability of the user to place a number of file markers in the recorded data file through control 30 to indicate some event in the experiment. A button 30 appears on the screens 10 and 50 that allows the user to place a marker in the data file to signify an event of his choosing. The file markers are placed in a separate column of data in the data file and are numbered sequentially starting at 1, which number is displayed to the user at text box 32. Because the data files are saved in continuous time increments, the file marker will be located in the file at the same temporal location that the event of interest occurred, and can therefore be correlated with the response to that event of the other parameters in their respective columns. Note that a place holder is required for each temporal location in the data file. The file marker column continues to record the current value of the file marker until a new one is chosen by the user. Buttons 30 for the file marker function are shown on the right, bottom of the screen or display 50 shown in FIGS. 4 and 5 discussed above. Also, on the graph 58 of Oxygen Saturation appear vertical blue lines 62 that indicate the temporal location of file marker's 1 and 2. The file marker location lines 62 can be supplied on each graph 52, 54, 56, 58 and 60.

Note that there are other ways to mark the data files other than a number. One could save a given type character that does not have to be a number, or a sequential integral number at that location, then keep all zeroes (or other character) at all other locations in the file marker column. File marking could also be done by having the user strike a key on the computer keyboard rather than or in addition to having a mouse click on a button on the user screen. This could also be done with a touch screen. In addition, it will be beneficial if textual comments can be added to each file marker either contemporaneously with the session or with a later review of the session.

Moving File Marker

As described above, an indicator 62 is placed on the graphs 52-60 described above to allow the user to see the time at which a given event was marked. This file marker indicator 62 appears as a vertical line on the screen as shown and as described, and it follows the time point on the graph 52-60 at which it was implemented until that time point leaves the sweeping visible screen in the future. Movement of the file markers is indicated by comparing the location of the vertical blue lines on the Oxygen Saturation plot between the figures above and below. The FIG. 5 shows the same run of data as the FIG. 4, except that it occurs some time later, as indicated by the movement of the vertical blue file marker lines 62 to the left (the screen scrolls from right to left). The curser data 44 time indication 42 is also indicative of a later time for the display 50 of FIG. 5.

Variable Display for Quick Diagnosis

A further concept is to display numerical values 20-28 of each parameter continuously during data collection, as described above for screens 10, 40 and 50. This utility allows the user to continuously see the actual numerical values associated with each scrolling graph. Additionally, the user can lay the computer mouse cursor over the plot at a given temporal location and left-click (or right click or the like). This will place all of the currently updated parameter values in boxes under the curser data heading 46 on display 50 adjacent to those that display the updating values for each parameter. A right click on the screen will load the current values into these adjacent boxes so that they do not update. This allows the user to take snapshots for review of all of the data parameters at a given time, allowing the device to be used as a diagnostic tool as well as a data recorder. This functionality is also available in the off-line data file review software.

User Adjustable Noise Suppression

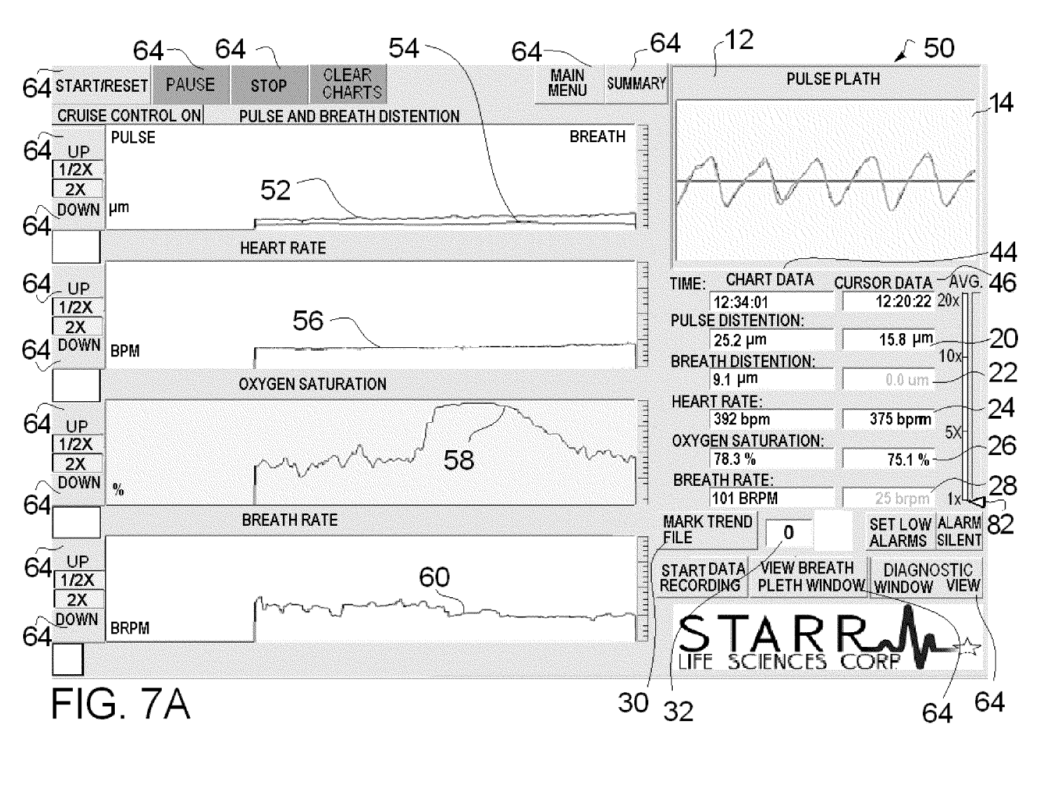

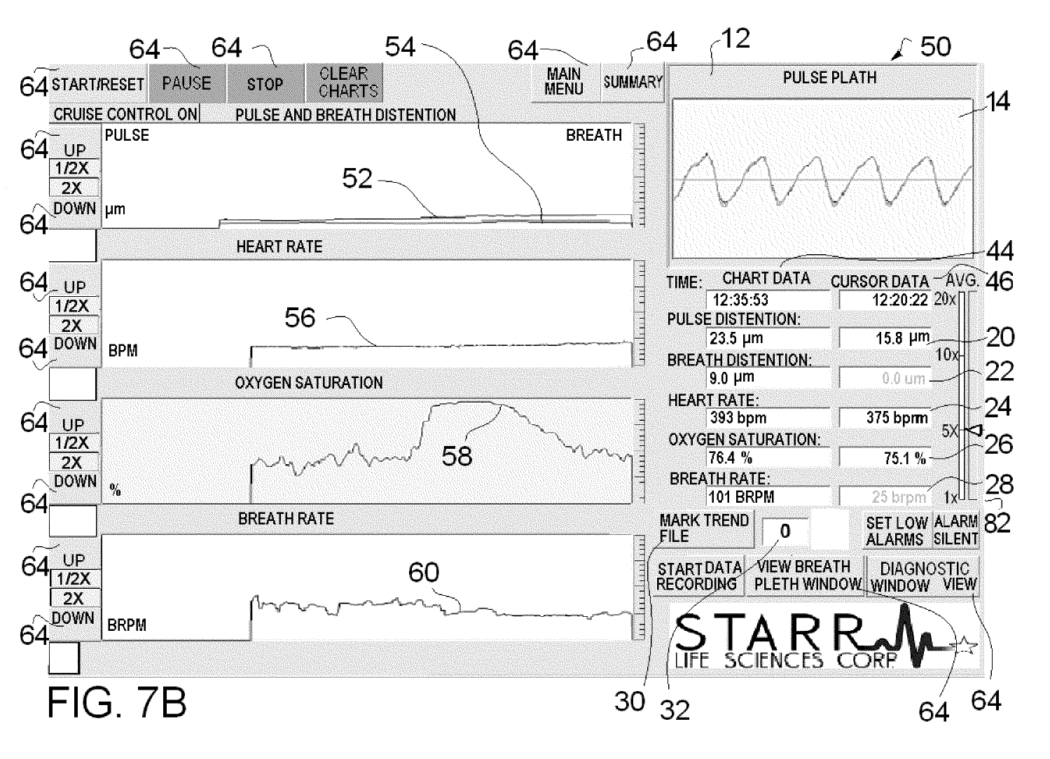

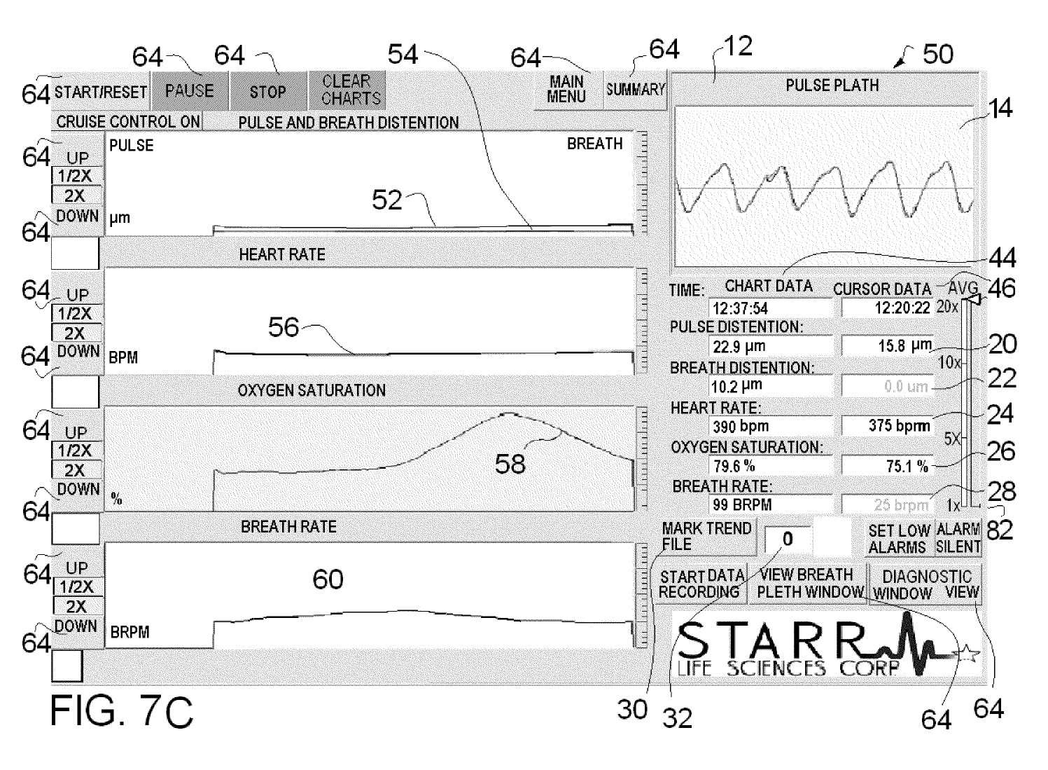

FIGS. 7A-C are representative illustrations of another version of a main data collection screen 50 with the addition of a user defined or adjustable noise suppression function. These figures represent three separate displays 50 of the same data file with three distinct settings for the user defined or adjustable noise suppression. The user adjustment is through the sliding control 82 on the right hand of the screen. Other controls for control 82 could be used such as a data input of a number for the amount of suppression. The sliding control 82 has proved to be an intuitively simple adjustment for the user. The purpose of this control 82 is to change the manner that each data point is calculated. Specifically the increase in this control 82 will increase the noise suppression. Specifically, increasing this value will increase the number of points that are used to calculate each data point on the graph. The system will move from 1× the standard set of data points to 20× the standard set of data points. The standard set of data points refers to the number of data points that the system uses to calculate a single final data point entry. For example, the system, as shown, uses a base or standard set of 10 data points for each final data point calculation such that the 20× listing will require 200 points. The effect of this control is to smooth out the function as the adjustment moves higher.

The noise suppression control identifies this function as averaging which helps convey the function to the user. However, averaging of the multiple or expanded data points is only one noise suppression or smoothing function that may be employed. A best fit line through the collection of data points (over time) can be used to identify the given data point, rather than a straight average of the values. Technically the average function can be viewed as a best fit horizontal line through the data points graphed over time. A higher order polynomial could be used with the data set. Further, weighted averages can be utilized by weighting of the data points (e.g. more recent carry more weight). The particular function utilized is transparent to the user and can be conveyed by the broad term “averaging” as shown.

Additionally it should be noted that the increasing of the data point set in the averaging function need not be always backwards in time. A data buffer would allow each calculation to include data points from before and after the given point. For a better understanding of this function, it is helpful to categorize the system data collection as collection a raw data set (e.g. each calculated heart rate) or data points; a display or calculated data set calculated from the raw data set, which is shown to the user and is in the system files as the data set; and a diagnostic data set described below which is an average of the calculated data set. The noise suppression adjustment allows the researcher to adjust the display or calculated date to be effective for his particular purpose for accuracy and responsiveness. In general the higher the noise suppression is set the more accurate each data point will be and the lower this is set the faster the system will respond to changes in the animal parameters. A review of FIGS. 7A-C, which are from the same raw data set, will better illustrate the results of this function.

Quick Diagnostic Screen

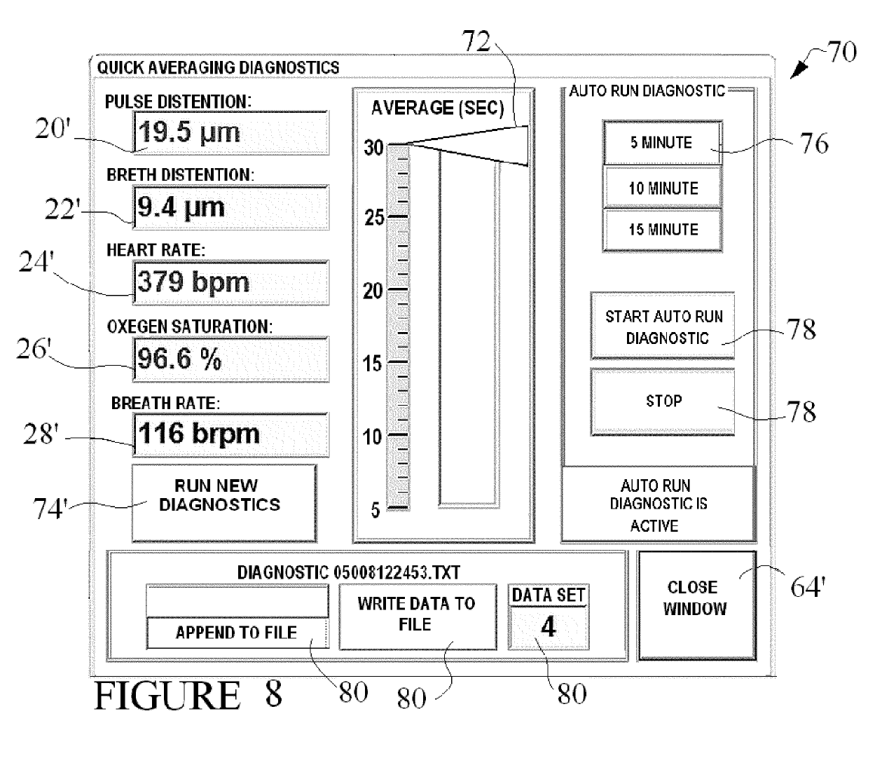

Another concept of the present invention is an addition to the diagnostic utility of the pulse oximeter device (see FIGS. 6 and 8), is a new user screen 70 that can be selected by a control button 64 on the data collection screen 50. This button 64 will pull up a new screen 70 shown in FIG. 6 that displays numerical values 20′, 22′, 24′, 26′ and 28′ of each of the data parameters. This screen 70 is designed specifically to allow the user to obtain single value data points which are averages of the data for quick diagnosis. The additional utility of this screen 70 is that it provides the user with the ability to select a period over which serial calculated data points are averaged for each parameter through controller 72. The user can then indicate when to start the count with control 74, and the software will average the selected serial data values over the chosen period set by controller 72 and display the final values when the average is completed in 20′-28′. The prime reference numerals are used as the values are averages of the selected parameter measurements rather than the measurements themselves. This averaging is done for each of the parameter (heart rate 24′, breath rate 28′, SₚO₂ 26′, pulse distention 20′, breath distention 22′ and any other obtained parameter). Note that the averaging period could also be set using particular quantities of updated values as well as the time-based approach given here. Note also that the averaging could be done either forward or backward in time (or both) from when the Run New Diagnostic button 74 is pressed. It should be noted that the averaging here can be classified as for data collection purposes for set data points and the “averaging” for the main screen is better classified as a noise suppression technique for processing the signal. The averaging here can also use the variety of data point averaging techniques, including a best fit line or higher order function, or a weighted average. However for data collection the simple average seems most appropriate and is a simple function to implement.

FIG. 8 illustrates an automated feature for the quick diagnostic feature. With this addition the user can select a time period in which the user can have the system automatically run with controls 78 and record the quick diagnostics at preset periods such as every 5, 10 or 15 minutes through controller 76. With this function selected the system will run and record the quick diagnostics at the specified times. For example, as shown the system will run 30 second averages on the data every 5 minutes and controls 80 are used to set the writing of the results to a data file in a designated manner.

Although the present invention has been described with particularity herein, the scope of the present invention is not limited to the specific embodiment disclosed. It will be apparent to those of ordinary skill in the art that various modifications may be made to the present invention without departing from the spirit and scope thereof. The scope of the present invention is defined in the appended claims and equivalents thereto.

What is claimed is:

1. A user interface for a pulse oximetry device that calculates physiologic parameters of a subject including at least a subject's heart rate and SₚO₂, the interface comprising a user selectable noise suppression setting that utilizes a designated multiple of data points for the calculation of at least one calculated physiologic parameters of the pulse oximetry device.

2. The pulse oximeter user interface of claim 1 further including a graphical display of a plurality of the calculated physiologic parameters over time.

3. The pulse oximeter user interface of claim 1 further including a recording of the calculated physiologic parameters and an event file marker function which is configured to be user selected to physically identify selected time locations of the record.

4. The pulse oximeter user interface of claim 1 further including a graphical display of a plurality of the calculated physiologic parameters over time, a numerical display of selected calculated physiologic parameters and an event file marker function which is configured to be user selected to physically identify selected time locations on the graphical display.

5. The pulse oximeter user interface of claim 1 wherein the noise suppression setting includes user selectable data averaging function in which the interface is configured to selectively obtain and display averages of at least some of the calculated physiologic parameters over a defined period.

6. The pulse oximeter user interface of claim 5 wherein the data averaging further includes a user selection of the defined average period.

7. The pulse oximeter user interface of claim 5 wherein the data averaging is configured to ignore calculated physiologic values that are deemed unacceptable in the calculation of the averages.

8. The pulse oximeter user interface of claim 5 wherein the data averaging is configured to display intermediate average values during calculation and to display final average values to the user in a distinct manner from the display of the intermediate average values.

9. The pulse oximeter user interface of claim 5 wherein the interface is configured to selectively begin the defined period at any time designated by the user.

10. The pulse oximeter user interface of claim 5 wherein the interface is configured to operate on recorded or real time data.

11. A user interface for a physiologic parameter sensor that calculates physiologic parameters of a subject, the interface comprising user selectable data averaging function in which the interface is configured to selectively obtain and display averages of at least some of the calculated physiologic parameters over a defined period.

12. The physiologic parameter sensor user interface of claim 11 wherein the data averaging further includes a user selection of the defined average period.

13. The physiologic parameter sensor user interface of claim 11 wherein the data averaging is configured to ignore calculated physiologic values that are deemed unacceptable in the calculation of the averages.

14. The physiologic parameter sensor user interface of claim 11 wherein the data averaging is configured to display intermediate average values during calculation and to display final average values to the user in a distinct manner from the display of the intermediate average values.

15. The physiologic parameter sensor user interface of claim 11 wherein the interface is configured to selectively begin the defined period at any time designated by the user.

16. The physiologic parameter sensor user interface of claim 11 wherein the interface is configured to operate on recorded or real time data.

17. The physiologic parameter sensor user interface of claim 11 further including a graphical display of a plurality of the calculated physiologic parameters over time and a user selectable event file marker function configured to physically identify selected time locations on the graphical display.

18. A user interface for a pulse oximetry device that calculates physiologic parameters of a subject including at least a subject's heart rate and SₚO₂, the interface comprising a graphical display of a plurality of the calculated physiologic parameters over time and a user selectable event file marker function configured to physically identify selected time locations on the graphical display.

19. The pulse oximeter user interface of claim 18 further including a user selectable data averaging function in which the interface is configured to selectively obtain and display averages of at least some of the calculated physiologic parameters over a defined period.

20. The pulse oximeter user interface of claim 18 further including a graphical display of at least one raw data signal of the pulse oximetry device that maintains heart and breath rate components.
